(19) United States
(12) Patent Application Publication
(10) Pub. No.: US 2007/0032805 A1
(43) Pub. Date: Feb. 8, 2007

(54) Oxydized cellulose prosthesis

(75) Inventors: Michel Therin, Lyon (FR); Alfred Meneghin, Lyon (FR); Jean-Louis Tayot, La Tour de Salvagny (FR); Suzelei Montanari, Trevoux (FR)

Correspondence Address: OLIFF & BERRIDGE, PLC, P.O. BOX 19928, ALEXANDRIA, VA, 22320, US

(73) Assignee: SOFRADIM PRODUCTION, Trevoux (FR)

(21) Appl. No.: 11/494,635

(22) Filed: Jul. 28, 2006

Related U.S. Application Data
(60) Provisional application No. 60/704,869, filed on Aug. 3, 2005.

(30) Foreign Application Priority Data
Mar. 16, 2006 (FR) 06/02316

(51) Int. Cl. A61F 2/02

(52) U.S. Cl. 606/151; 623/23.74; 623/23.75

(57) ABSTRACT

The present invention relates to a three-dimensional prosthetic fabric comprising a first face and a second face, the said first face and the said second face being opposite each other and separated by the thickness of the said fabric, the said first face being porous, characterized in that the said second face is dense and made of at least one first resorbable yarn. The invention also relates to a process for preparing such a fabric and to a prosthesis obtained using such a fabric.

The present invention relates to a three-dimensional prosthetic fabric that is useful especially for obtaining reinforcing prostheses in parietal and/or visceral surgery, and that is particularly suitable for preventing post-surgical adhesions in case of a use via the intraperitoneal path.

Post-surgical adhesions include all non-anatomical fibrous bonds fortuitously induced by a surgical act during the normal cicatrisation process. They may occur in any field of surgery, irrespective of the action under consideration. They are generally all the more severe the greater the surgical trauma and the more effected the tissues that normally provide the planes of cleavage (intersticial connective tissue, synovial tissue, tendon sheaths, peritoneal and pleural serous tissue, etc.). Any surgical trauma to a tissue is followed by a cascade of physiological events, the main times of which may be simplified as follows:
time zero (t0): surgical trauma, capillary effraction;
time zero plus a few minutes: clotting, formation of the fibrin network, release of the chemotactic factors;
time zero (t0) plus 12 to 48 hours: leukocyte afflux with a predominance of polymorphonuclear leukocytes;
time zero (t0) plus 24 hours to 5 days: leukocyte afflux with a predominance of macrophages;
time zero (t0) plus 4 to 8 days: fibroblast afflux;
time zero (t0) plus 5 to 14 days: connective differenciation of the cicatricial reaction;
time zero (t0) plus 15 to 180 days: cicatricial remodelling.

Although the exact mechanisms are for some of the events still unknown, especially as regards the determinism of the intensity of the reaction, it thus appears that the first days are determining since they condition the fibroblast afflux responsible for the formation of adhesions.

As a result, such post-surgical adhesions may cause syndromes that may be classified mainly as chronic pain, occlusive syndromes, and female infertility. Moreover, they very substantially increase the risks of false routes during a reintervention (myocardial or intestinal effraction during iterative thoracotomy or laparotomy), while at the same time prolonging the operating times, the preliminary dissection possibly being very laborious in such cases.

Moreover, in visceral and parietal surgery, the reinforcing prosthesis needs to have a certain amount of mechanical strength to allow it to fulfil its function as a surgical reconstruction component. In general, the prosthetic fabrics known especially in the treatment of parietal insufficiency, for example hernias and ventral ruptures, provide additional mechanical strength to the surgical reconstruction. Such fabrics are all the more efficient and their local tolerance is all the better when their tissue integration is intimate and early. For this reason, prosthetic fabrics that are particularly efficient in these indications are three-dimensional fabrics with large porosity, and are designed so as to be integrated into the body as quickly as possible

Such openwork three-dimensional prosthetic fabrics are described, for example, in WO 99/05990.

In an attempt to overcome the problem of visceral surgical adhesions following an operation for implantation of such a reinforcing prosthesis in an intraperitoneal site, it has been proposed to place a physical barrier between the said three-dimensional prosthetic fabric of the reinforcing prosthesis and the adjacent organic structures whose adhesion it is desired to prevent. However, the desired barrier effect poses the problem of the intrinsic adhesion-generating capacity of this barrier. The reason for this is that, if the barrier consists of a non-resorbable material, it may itself be the cause of adhesions over time; and if it is resorbable, its resorption should cause sufficiently little inflammation so as not to give rise, itself, to adhesions.

In particular, to avoid the latter phenomenon, it has been proposed to cover one face of the reinforcing prosthesis with a smooth, non-porous material so as not to offer any space for cellular recolonization. Thus, in WO 99/06079 and WO 99/06080, an openwork three-dimensional prosthetic fabric covered on one of its faces with a polysaccharide-based or collagen-based smooth, non-porous resorbable film, has been proposed.

However, the manufacture of such prostheses requires a combination of two different steps, one following the other, namely, in a first stage, the preparation of the three-dimensional textile structure, and then, in a second stage, the treatment of one face of the textile prosthesis in order to render it smooth and non-porous. A method for treating this face consists in soaking the textile prosthesis in a hydrogel in order to impregnate the said face therewith. The assembly is then subjected to a drying operation, in order for the hydrogel to be converted into a smooth, non-porous, continuous dry film.

Alternatively, a composite prosthesis may be made, combining a textile face with a non-stick polymeric face, which may or may not be resorbable. In these composite prostheses, the two faces, i.e. the textile face and the polymeric face, are bonded together.

Irrespective of the treatment method used, it has disadvantages: long and laborious in the first case, inefficient in the second case due to the fragility and the instability of the textile face-polymeric face bond in the case of the composite prosthesis.

Thus, there is a need for a reliable reinforcing prosthesis that is simple and easy to manufacture, which firstly has mechanical properties sufficient to ensure its role of reinforcing a tissue wall, but which, secondly, limits, or even prevents, the development of post-surgical adhesions on its face that is in contact with the viscera.

The present invention is directed towards remedying this need by proposing a three-dimensional fabric, in particular made of a single component, having a porous face to promote cellular colonization, and a resorbable dense face.

The present invention relates to a three-dimensional prosthetic fabric comprising a first face and a second face, the said first face and the said second face being opposite each other and separated by the thickness of the said fabric, the said first face being porous, characterized in that the said second face is dense and is made of at least one first resorbable yarn.

The fabric according to the invention is particularly suitable for use as a reinforcing prosthesis in parietal and visceral surgery.

The present invention also relates to a prosthesis for reinforcing, protecting or supporting a tissue wall, characterized in that it is obtained by cutting out from a prosthetic fabric as described above.

The fabric and/or the prosthesis according to the invention allow the fastest possible tissue integration of the face which is in contact with the wall to be reinforced, affording mechanically satisfactory anchoring, without extensive fibrosis, which is the cause of discomfort or pain, while at the same time preventing the formation of post-surgical adhesions on contact with the organs surrounding the visceral or intraperitoneal face.

Moreover, due to the resorbable nature of the dense face of the fabric and/or prosthesis according to the invention, the amount of non-resorbable material is reduced and any potential chronic inflammatory reaction on contact with the said non-resorbable material is thus limited. Thus, the prosthesis according to the invention has considerably reduced long term intrinsic inflammatory capacity compared with any other standard prosthesis not comprising a resorbable dense face.

In the present patent application, the term “fabric” means an assembly or arrangement of monofilament or multifilament yarns, obtained by knitting and/or weaving.

In the present patent application, the term “prosthetic fabric” means a fabric intended to be implanted into the human or animal body in the form of a prosthesis or of any other component made at least partly with the said fabric.

In the present patent application, the term “three-dimensional fabric” means a fabric having a significant thickness, preferably of greater than or equal to 0.5 mm.

In the present patent application, the term “porous face” means a face whose surface has a certain level of coarseness, for example alveolae, holes or orifices, opening in its surface, which may or may not be uniformly distributed, promoting any cellular colonization. The porous face is the face of the prosthetic fabric intended to come into contact with, and then to be integrated with, the tissue wall to be reinforced or protected.

In the present patent application, the term “dense face” means a face that has in places porous faces, but whose overall surface generally shows a certain level of unity and homogeneity. This dense face is intended to be exposed to the viscera adjacent to the tissue wall, during the repair or regeneration of said tissue wall.

In the present patent application, the term “resorbable” means the characteristic according to which a material is absorbed by the biological tissues and disappears in vivo after a given period, for example within 3 months, or alternatively within 4 weeks, or alternatively within a few days.

In the present patent application, the term “rib” means the ply or plies that link(s) together the two faces of a three-dimensional fabric, thus constituting the thickness of such a fabric.

According to the present invention, the dense face is made of a first resorbable yarn. This or these resorbable yarn(s) is (are) capable of becoming partially or totally transformed in vivo, on contact with organic tissues and their secretions, into a continuous hydrogel capable of serving the purpose of preventing adhesions.

Preferably, the said first resorbable yarn consists of monofilaments and/or multifilaments of a biocompatible polymer material chosen from polyesters, polycaprolactones, polydioxanones, polyarnides, polyethers and polysaccharides, and mixtures thereof.

The polyesters may be chosen from polyhydroxy acids, preferably glycolic acid polymers, lactic acid polymers or hydroxybutyric acid polymers, and mixtures thereof.

The polysaccharides may be chosen from hyaluronic acid, alginic acid, polyglucuronic acid, chitosan, starch, soluble cellulose derivatives, their salts and mixtures thereof. Said polysaccharide may be crosslinked.

The polyglucuronic acid may originate from bacteria, like the polysaccharide secreted by the mutant strain of Rhizobium meliloti M5N1CS(NCIMB 40472), according to the teaching of WO9318174, or it may be obtained by selective oxidation of primary hydroxyl groups of cellulose.

The soluble cellulose derivatives may be chosen from cellulose ethers, for instance carboxymethylcellulose, and oxidized celluloses, and mixtures thereof.

Preferably, the oxidized celluloses are chosen from oxidized cellulose in which the C₆ primary alcohol is partially or totally oxidized to a carboxylic acid, for example, to give polyglucuronic acid, cellulose oxidized in the form of polyaldehydes with periodic acid, and cellulose of “viscose” type, manufactured from a solubilized and then regenerated and oxidized cellulose pulp, and mixtures thereof.

Several varieties of regenerated cellulose have been developed industrially. Examples that may be mentioned include the “viscose” process, which is based on the solubility of cellulose xanthate in dilute sodium hydroxide solution. Mention may also be made of the “cuproammonium process” used, for example, by the company Bemberg in Italy or the company Asahi Chemical Industries in Japan, and which consists in dissolving cellulose in an ammoniacal copper solution. Another process for preparing regenerated cellulose that is suitable for the present invention is the process of dissolving cellulose in an organic phase with N-methyl-morpholine oxide (NMMO), which is known as the “Lyocell® process”, used, for example, by the company Lenzing in Austria.

When threaded through a perforated plate, viscose coagulates in acidic medium and forms long continuous filaments of regenerated cellulose, which are dried and combined as multifilament yarns. A regenerated cellulose yarn that has good mechanical strength is obtained.

In general, such a regenerated cellulose yarn is not resorbable. Thus, as will be described later in the present patent application, preferably, the dense face of the fabric according to the invention will be made in a first stage with such a regenerated cellulose yarn, and this dense face will then be subjected in a second stage to an oxidation process in order to render the said regenerated cellulose yarn resorbable.

An example that may be mentioned of a regenerated cellulose yarn that is suitable for the present invention is the 90 decitex multifilament yarn sold under the name “CUPRO® Cusio” by the Italian company Bemberg.

In one embodiment of the invention, the said first resorbable yarn is a regenerated and oxidized cellulose multifilament yarn.

In another embodiment of the invention, the said first resorbable yarn is a composite multifilament yarn of polyglycolic acid and of oxidized cellulose in the form of polyglucuronic acid.

In another embodiment of the invention, the said first resorbable yarn is a chitosan yarn, or a crosslinked hyaluronic acid yarn. Such a crosslinked hyaluronic acid yarn is based on a polymer made with a low degree of crosslinking so that it can become rapidly hydrated and can degrade in less than four weeks.

In yet another embodiment of the invention, the said first resorbable yarn is obtained by mixing a negatively charged polysaccharide, chosen from alginic acid, hyaluronic acid, polyglucuronic acid and mixtures thereof, and a positively charged polysaccharide, for instance chitosan.

According to one embodiment of the invention, the said first resorbable yarn is a multifilament yarn composed of multiple filaments defining between themselves interstitial spaces, and the said multifilament yarn is impregnated, in the said interstitial spaces, with a polysaccharide chosen from hyaluronic acid, alginic acid, polyglucuronic acid, chitosan, starch and soluble cellulose derivatives, and mixtures thereof.

Impregnation with a viscous solution of polysaccharides is preferably performed by passing the dry yarns in a bath of the solution or several successive baths of different solutions. On removal, the yarns may be dried directly before being reeled. The drying phase may be proceeded by a phase of coagulation of the polysaccharides in a volatile solvent such as acetone or isopropanol. This solvent may at the same time provide an agent for crosslinking the polysaccharide chains, such as a difunctional reagent of diepoxide type, for instance butanediol diglycidyl ether, which will react with the hot polysaccharide during the drying phase. In one particular embodiment of the invention, crosslinking of the polysaccharides is obtained via natural electrostatic bonds between the opposite charges of two mixed polysaccharides of opposite charge and preferably by successive deposition of a first layer of positively charged chitosan, followed by a second layer of negatively charged polysaccharide, like for instance hyaluronic acid.

By means of this impregnation, the yarn becomes highly hydrophilic at the surface and the chosen polysaccharide will be rapidly released or hydrated in the presence of moisture from the biological tissues, or by wetting the prosthetic fabric of the invention before implanting it in the patient, creating a viscous gel anchored in the mesh of the fabric, by means of its viscosity and its crosslinking, if any. The spontaneous creation of this continuous gel gives the fabric according to the invention adhesion-preventing properties.

The resorbable nature of the dense face of the fabric according to the invention, in particular when the resorbable yarns of constituting this dense face are impregnated with polysaccharides as described above, makes it possible to convert a discontinuous textile face, for example made by knitting, into a continuous gelled face, by a wetting previously to implantation or by simply placing in contact with the biological tissues to be repaired and protected.

Thus, the said first resorbable yarn may be a multifilament yarn of polyglycolic acid impregnated with polyglucuronic acid.

In another embodiment of the invention, the said first resorbable yarn is a multifilament yarn of polyglycolic acid impregnated with a mixture of polyglucuronic acid and chitosan.

In another embodiment of the invention, the said first resorbable yarn is a multifilament yarn of polyglycolic acid impregnated with a mixture of hyaluronic acid and chitosan.

In another embodiment of the invention, the said first resorbable yarn is a multifilament yarn of polyglycolic acid impregnated with hyaluronate crosslinked with 1,4-butanediol diglycidyl ether.

In another embodiment of the invention, the said first resorbable yarn is a multifilament yarn of oxidized cellulose impregnated with chitosan.

According to one embodiment of the invention, the said first porous face is made of at least one second resorbable or non-resorbable yarn, consisting of monofilaments and/or multifilaments of a biocompatible polymer material.

Preferably, the said second yarn is non-resorbable. The mechanical reinforcing function of the fabric is thus definitively ensured and the risks of recurrences of hernias due to resorption of the implant are avoided.

According to one embodiment of the invention, the said second yarn consists of monofilaments and/or multifilaments of a biocompatible polymer material chosen from polypropylene, polyethylene terephthalate, polytetrafluoroethylene, polyarnide, polyvinyldifluorene and mixtures thereof.

In another embodiment, the porous face is made of at least one second resorbable yarn, for example of polylactic acid. This case is particularly suitable when the risks of recurrence are low or when the quality of the tissue regeneration may be optimal.

According to one embodiment of the invention, the said first face and the said second face are linked together via a rib made of at least one third resorbable or non-resorbable yarn, consisting of monofilaments and/or multifilaments of a biocompatible polymer material.

Preferably, the said third yarn is resorbable. This case is particularly preferred when the reinforcing function may be fulfilled by only one non-resorbable porous face.

Thus, preferably, the said third yarn is resorbable and consists of monofilaments and/or multifilaments of a biocompatible polymer material chosen from polyesters, polycaprolactones, polydioxanones, polyalkanoates, polyamides, polyphosphazenes, polyacetals, polyurethanes, polyorthoesters, polycarbonates and polyanhydrides, and mixtures thereof.

The polyesters may be chosen from polyhydroxy acids, preferably from glycolic acid polymers, lactic acid polymers and hydroxybutyric acid polymers, and mixtures thereof.

In one embodiment of the invention, the said third yarn is a polylactic acid multifilament yarn.

In another embodiment of the invention, the said third yarn is a polyglycolic acid multifilament yarn.

In yet another embodiment of the invention, the said third yarn is a polylactic and glycolic acid multifilament yarn.

In another embodiment, the said third yarn is non-resorbable. This is the case, for example, for wall surfaces and/or volumes to be repaired that are very large or that are subjected to higher than average anatomical tensions. The regenerated wall is thus permanently reinforced by a non-resorbable fabric that is thicker than the porous face alone.

According to one embodiment of the invention, the said dense face comprises at least one lap of close-knit first yarns which determines a unified but permeable face. The close knitting of the resorbable yarns of the dense face allows the production of a continuous hydrogel, by aqueous impregnation of the fabric or prosthesis of the invention before implantation, or on contact of these yarns with the natural moisture of the organic tissues, once the fabric or the prosthesis of the invention is implanted.

According to another embodiment of the invention, the said porous face comprises at least two laps of knitted second yarns, determining hexagonal-shaped apertures.

In one embodiment of the invention, the said rib comprises at least one lap of third yarns, extending substantially perpendicularly from the porous face to the dense face, the said third yarns being distributed, on the said porous face, along the peripheral edges of the said hexagonal apertures.

Preferably, the said third yarns form substantially parallel transverse channels, the internal cross section of which is free of yarns, the said channels emerging on either side of the fabric, on the porous face according to the said hexagonal apertures, and on the dense face, respectively.

Preferably, the mean diameter of the transverse channels is greater than or equal to 0.3 mm, preferably ranging from 0.7 to 3 mm and more preferably ranging from 1.3 to 1.7 mm.

Preferably, the transverse channels have a length, corresponding to the thickness of the said fabric, ranging from 0.5 to 5 mm and more preferably ranging from 1.5 to 3 mm.

Preferably, the rib determines for each channel a porous inner wall interconnecting with the neighbouring channels, the said porous inner wall defining interstices for passing between channels.

Advantageously, the said interstices for passing between channels have a width ranging from 100 to 300 microns.

The porosity of the wall of the channels is determined in particular by the textile arrangement of the said third yarns, which may have, for example, a diameter of between 10 and 15 μm.

The walls of the channels provide an anchoring area for the fibrous reaction under the control of the prosthesis (immediate environment of each yarn), which contributes, however, towards relatively intimate and early tissue integration of the prosthetic fabric. Furthermore, when the internal cross section of each alveolus or channel is substantially free of any connecting yarn, the inflammatory reaction of the prosthetic fabric in vivo is proportionately reduced, thus limiting the formation of peripheral fibrous capsule responsible for secondary cicatricial contraction. This highly porous three-dimensional structure of the fabric according to the invention allows differentiation of a histologically normal connective tissue at the core of the prosthesis. The multidirectional porosity also promotes drainage of the site and thus limits the risks associated with accumulations of fluids (seromas, haematomas, sepsis).

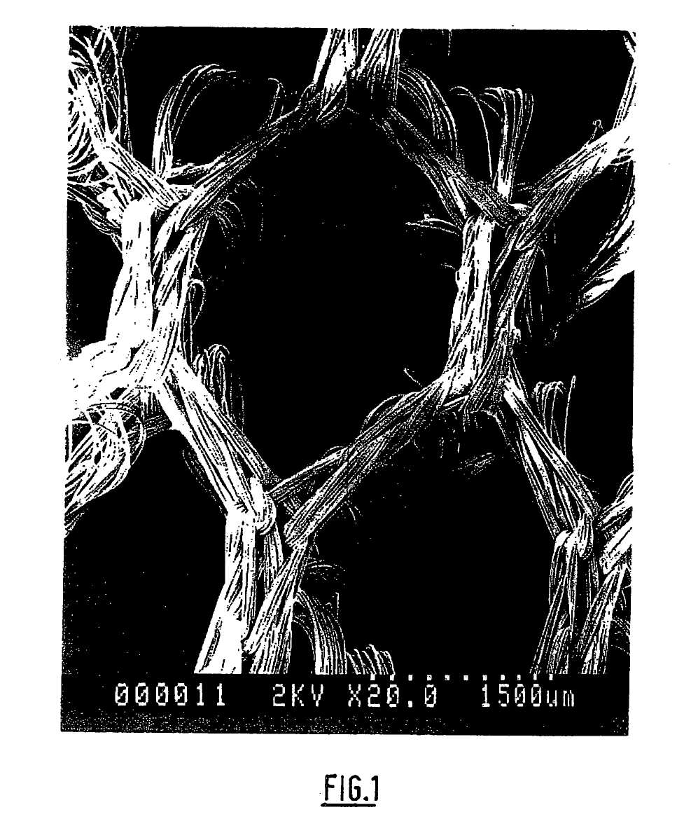

Once the prosthetic fabric has been implanted, the cells present at the centre of the volume created by the three-dimensional structure are at least 750 μm away from any prosthetic material, if the size conditions defined above are respected, and as shown later in FIG. 1 accompanying the present description. Thus, the colonizing cells are far from any influence that might delay or disrupt the differentiation mechanisms, while at the same time being less than one millimetre from the receiving tissue, i.e. close to elements providing the elements that are essential for rapid rehabitation (progenitor stem cells, blood capillaries, etc.).

These conditions make it possible to obtain mechanically satisfactory anchoring while at the same time preserving differentiation achieved at the core, as encountered in normal connective tissue. When this rib consists of resorbable yarns, any inflammatory reaction will have disappeared after resorption of these yarns. The regenerated connective tissue remains stable, provided that it has been able to grow and differentiate in the porous architecture of the prostheses according to the invention.

By means of the architecture of the space created by the fabric and/or the three-dimensional prosthesis according to the invention, and especially the dimension of the pores and their interconnections, the fabric and/or the prosthesis according to the invention allow optimum cellular colonization and tissue integration.

Furthermore, once the fabric or the prosthesis according to the invention is implanted, regeneration tissue progressively grows on the dense face, in contact with the regenerated tissue in the porous face and the rib. This regeneration tissue recreates a covering tissue leaflet, which is well-structured and stable, even after degradation of the resorbable dense face. This tissue leaflet definitively distances any adjacent organ from the non-resorbable part of the fabric or the prosthesis thus limiting the risks of post surgical visceral adhesions.

Moreover, since the dense face of the fabric or prosthesis according to the invention forms an integral part of the said fabric or of the said prosthesis, before implantation, this dense face is entirely stable and presents no risk of becoming separated or coming away from the said fabric or the said prosthesis, as, for example, in the case of composite prostheses manufactured in two steps, for instance by glueing.

In the fabrics and prostheses according to the invention, the said first, second and third yarns may be identical or different. In particular, the fabric and the prosthesis according to the invention may be fully resorbable, for example in the case where the tissue wall reinforcing function of the prosthesis is desired only for a temporary period.

In general, however, the reinforcing function of the prosthesis will be desired permanently and the said second yarns, constituting the porous face, will be different from the said first yarns constituting the dense face, and will be non-resorbable. In this case, the said third yarns, constituting the rib, may be non-resorbable, and identical to or different from the said second yarns, or alternatively resorbable, and identical to or different from the said first yarns constituting the dense face.

Finally, the fabric and prostheses of the invention are particularly quick and easy to manufacture. Indeed, according to a first embodiment of the invention, the process for preparing the fabric and prostheses of the invention may be performed in a single step, for example by knitting or weaving, and this process requires no specific operation for treating one face of the fabric in order to make this face resorbable. This is particularly true when one may choose, before knitting or weaving, a simple or composite resorbable yarn so as to constitute the dense face.

According to a second embodiment of the invention, the process for preparing the fabric and prostheses of the invention comprises a first step of fabric manufacturing, for instance knitting or weaving, and then a subsequent step of oxidation of the fabric. In such a case, it is possible to choose, for the yarn intended to serve as the constitutive yarn for the dense face, a yarn which is not resorbable before oxidation, and becomes resorbable after oxidation. It is for example the case when one chooses a yarn made of regenerated cellulose, for example non oxidized, for the yarn intended to serve as the constitutive yarn for the dense face. The yarn made of non oxidized regenerated cellulose becomes resorbable after an oxidation step.

In all cases, the formation of a continuous film of hydrogel, at the level of the dense face of the fabric and prostheses of the invention, as seen above, does not require any specific manufacturing step: this film is formed by simple wetting of the fabric or prosthesis of the invention before implantation, or when the fabric or prosthesis of the invention is in contact with the aqueous secretions of the organic tissues to be protected.

Another subject of the invention relates to a process for preparing a three-dimensional prosthetic fabric comprising a first face and a second face, the said first face and the said second face being opposite each other and separated by the thickness of the said fabric, the said first face being porous, the said second face being dense and resorbable, comprising a step of manufacturing a three-dimensional knitted fabric on a warp knitting machine or Raschel knitting machine in at least one lap of yarns defining the porous face, at least one lap of yarns defining the thickness of the said fabric, and at least one lap of yarn defining the said dense face, characterized in that the said lap defining the said dense face is obtained using a full-threaded guide bar with at least one first resorbable yarn.

The fabric may then be stabilized simply by baking at a temperature of between about 80° C. and 150° C.

Yet another subject of the invention relates to a process for preparing a three-dimensional prosthetic fabric comprising a first face and a second face, the said first face and the said second face being opposite each other and separated by the thickness of the said fabric, the said first face being porous, the said second face being dense and resorbable, comprising a step of manufacturing a three-dimensional knitted fabric on a warp knitting machine or Raschel knitting machine in at least one lap of yarns defining the porous face, at least one lap of yarns defining the thickness of the said fabric, and at least one lap of yarn defining the said dense face, characterized in that:

in a first step, the said lap defining the said dense face is obtained using a full-threaded guide bar with at least one first regenerated cellulose yarn, and

in a second step, the said fabric is subjected to an oxidation step.

In one embodiment of the invention, the said fabric is subjected to oxidation with sodium metaperiodate or with periodic acid, and is then rinsed in an aqueous acetone or alcohol solution, and washed with pure acetone or alcohol before drying. For example, said fabric may be subjected to oxidation with 10 mM periodic acid, for 15 hours at room temperature.

In another embodiment of the invention, the said fabric is subjected to oxidation with nitrogen dioxide (NO₂) in a suitable non-aqueous solvent and is then washed with an aqueous isopropanol solution. It is possible to use the NO₂ in a gas state, alone without any other liquid or gaseous solvent. Preferably, said fabric is subjected to oxidation with gaseous nitrogen dioxide at a temperature ranging from 20 to 50° C., in particular from 25 to 40° C., for example for a time ranging from 2 to 48 hours, in particular from 3 to 8 hours. In order to avoid the condensation of nitrogen dioxide, it is preferred to use a nitrogen dioxide concentration less than 14 g/L, in particular ranging from 6 to 12 g/L. It is preferred that the weight ratio NO₂/cellulose be greater than 0.9 in order to ensure a sufficient oxidation. Alternatively, it is possible to realise the oxidation with nitrogen dioxide in the presence of air or oxygen. Alternatively, it is possible to use NO₂ in a gas such as CO₂ or nitrogen or in chlorinated or perfluorinated liquid solvents, for instance carbon tetrachloride, freons or substitution products thereof, according to the teaching of patent U.S. Pat. No. 3,364,200. It is also possible to use NO₂ in a gas like CO₂ or nitrogen, maintained in a dense or supercritical state as disclosed in the application WO20060118552.

Preferably, after oxidation with nitrogen dioxide, said fabric is subjected to a washing step with an inert gas like CO₂ or N₂ in order to remove the excess of NO₂, followed by a washing with a volatile alcohol. For example, the volatile alcohol is pure isopropanol.

Preferably, a plasticizer, such as glycerol or polyethylene glycol, is added to the washing step.

In one embodiment of the invention, the said first yarns of the lap defining the dense face are knitted according to the scale 1112/1110//.

In another embodiment of the invention, the said first yarns of the lap defining the dense face are knitted according to the scale 2223/1110//.

According to one embodiment of the invention, before oxidation, the fabric is stabilized by baking at a temperature ranging from 80 to 150° C.

The various embodiments and the advantages of the present invention will now emerge from the attached drawings, in which:

FIG. 1 is a view obtained with a scanning electron microscope “HITACHI type S 800” at magnification ×20, of the porous face of a fabric according to the invention,

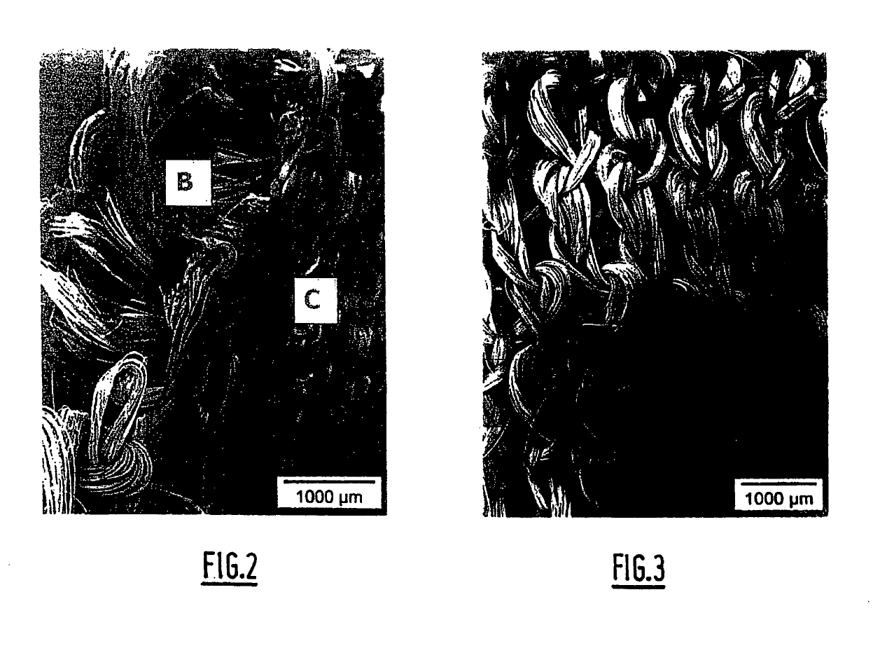

FIG. 2 is a view obtained with a scanning electron microscope “HITACHI type S 800” at magnification ×30, of the rib and of the dense face of a variant of the fabric according to the invention,

FIG. 3 is a view obtained with a scanning electron microscope “HITACHI type S 800” at magnification ×30, of the dense face of a the fabric of FIG. 2,

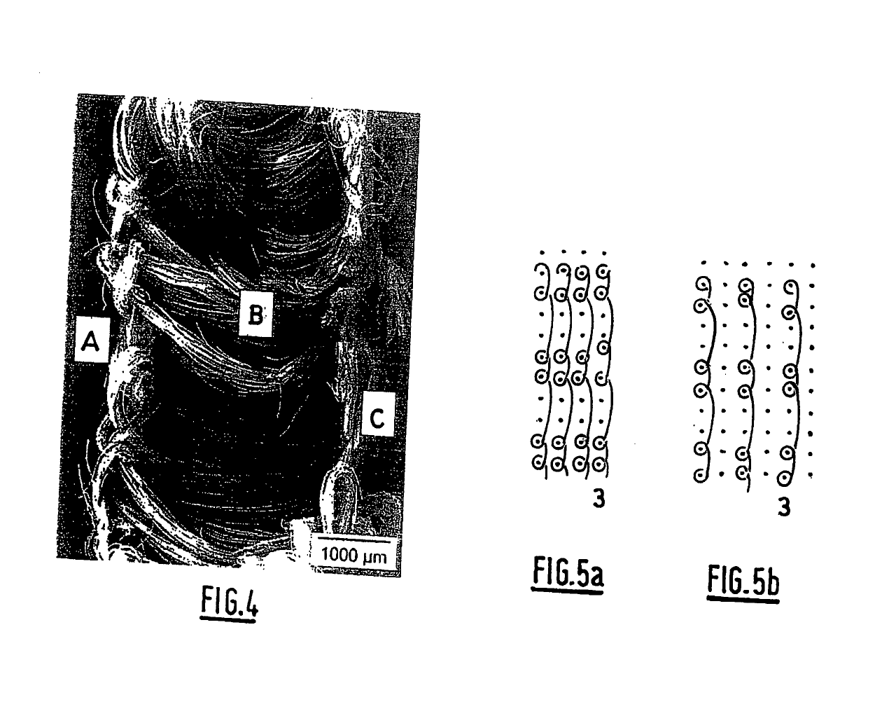

FIG. 4 is a view obtained with a scanning electron microscope “HITACHI type S 800” at magnification ×30, of a vertical cross section of the porous face, of the rib and the dense face of the fabric of FIG. 2,

FIGS. 5a and 5b are schematic drawings of two variants of knitting weaves to obtain the rib of a fabric according to the invention,

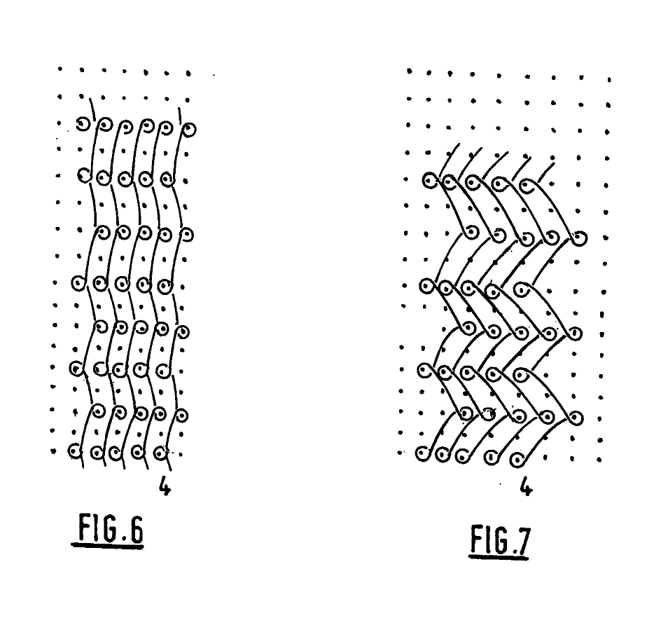

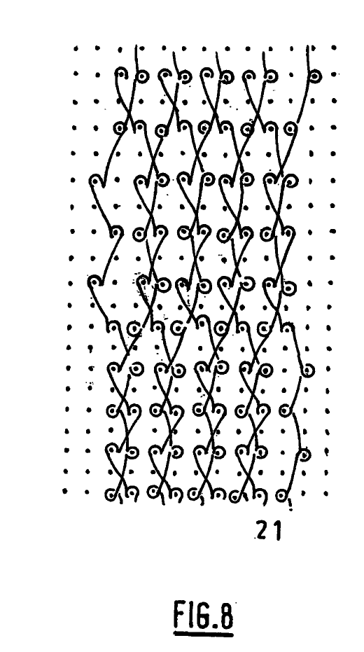

FIGS. 6 and 7 are schematic drawings of two variants of knitting weaves to obtain the dense face of a fabric according to FIGS. 2-4,

FIG. 8 is a schematic drawing of a knitting weave to obtain the porous face of a fabric according to FIG. 1.

FIG. 1 shows an example of a porous face of a three-dimensional prosthetic fabric according to the invention. According to this figure, the porous face is independent and has hexagonal-shaped apertures. The apertures of this face are defined by peripheral edges, formed with the constituent yarns of this face. Preferably, these yarns are non-resorbable so as to ensure the permanent tissue wall reinforcing function of the fabric or prosthesis. When this reinforcing function is desired only for a determined time, these yarns may be resorbable. In the embodiment shown in FIG. 1, the constituent yarn of the porous face is a 50 decitex multifilament polyester yarn.

The porous face shown in FIG. 1 is knitted on a double bed Raschel knitting machine with two laps of yarns (1, 2), according to the knitting weave shown in FIG. 8, according to a representation scheme that is standard to those skilled in the art and that will not be described in further detail herein. The needle bars of the knitting machine, corresponding to yarns 1 and 2, are threaded one full-one empty. According to the weave described in FIG. 8, the yarns are knitted according to the following scales:
1211/1011/1211/1011/1222/3222/1222/3222// for the lap of yarns 1,
1222/3222/1222/3222/1211/1011/1211/1011// for the lap of yarns 2.

As is clearly seen in FIG. 1, such knitting gives a porous face with hexagonal apertures with a mean diameter ranging from about 1.3 mm to 1.7 mm. Such a porous face is thus entirely favourable towards good cellular colonization and good tissue integration. The reason for this is that, as indicated, once the prosthetic fabric is implanted, the cells, present at the centre of the volume created by the three-dimensional structure, are thus at least 750 μm away from any prosthetic material. Thus, the colonizing cells are far from any influence that might delay or disrupt the differentiation mechanisms, while at the same time being less than one millimetre away from the receiving biological tissue, which represents optimum conditions for obtaining mechanically satisfactory anchoring while at the same time preserving differentiation achieved at the core, as encountered in normal connective tissue.

On FIGS. 2-4 is shown a fabric of the invention for which the porous face (A) is realised according to the same method as the porous face of FIG. 1, but with a polypropylene monofilament yarn of diameter 0.1 mm: such a yarn is commercially available under the name “CRINLENE®” by the Italian company SIDER ARC.

As may be seen in FIGS. 2 and 4, the porous face (A) and the dense face (C) are connected together via the rib (B), which, in FIGS. 2 and 4, comprises a lap of yarns, also known as the intermediate bonding lap, which extends substantially perpendicularly from the porous face (A) to the dense face (C). The constituent yarns of this bonding lap are distributed along the peripheral edges of the hexagonal apertures of the porous face. The bonding yarns thus distributed form substantially parallel transverse channels, the internal cross section of which is free of yarns. These transverse channels emerge on either side of the fabric, on the porous face and the dense face, respectively.

In accordance with the invention, the bonding yarns are arranged such that each transverse channel or alveolus has a porous inner wall for lateral interconnection with the neighbouring channels, these interstices having a diameter of between 100 and 300 μm. The transverse channels increase the rate of cellular colonization, once the fabric has been implanted in vivo, since they facilitate the conveyance of cells or the cellular afflux to the site of the implantation. Moreover, the virtual absence of yarns in the very volume of the transverse channels makes it possible to reduce the inflammatory reaction caused by the prosthetic fabric, which further favours good implantation thereof.

The rib shown in FIGS. 2 and 4 is knitted on a double bed Raschel knitting machine with a lap of yarns 3, according to the knitting weave shown in FIG. 5a, according to a representation scheme that is standard to those skilled in the art. The yarn which is used is a regenerated cellulose multifilament yarn 90 decitex, commercially available under the name “CUPRO® Cusio” by the Italian company Bemberg. The needle bar of the knitting machine, corresponding to the yarns 3, is full-threaded as shown in FIG. 5a. In another embodiment of the invention, this needle bar may be threaded one full-one empty as shown in FIG. 5b. In yet another embodiment of the invention, this needle bar may be threaded with an irregular full and empty arrangement. According to the weaves described in FIGS. 5a and 5b, the yarns are knitted according to the following scale: 0101/0000//.

FIG. 3 shows the dense face of a fabric according to the invention. As is seen in this figure, this dense face may be independent, and it has a unified, dense but, however, permeable face. The constituent yarns of this dense face are resorbable and preferably close-knitted, as seen in FIG. 3. This close knitting and the resorbable nature of these yarns make them capable, upon a wetting previous to implantation or on contact with organic tissues in vivo, of converting this dense face into a hydrogel capable of ensuring the adhesion-preventing function.

The dense face shown in FIG. 3 is knitted on a double bed Raschel knitting machine with a lap of yarns 4, according to the knitting weave shown in FIG. 6, according to a representation scheme that is standard to those skilled in the art. The yarn which is used for the knitting step is the same as that of the rib, namely a regenerated cellulose multifilament 90 decitex yarn, commercially available under the name “CUPRO® Cusio” by the Italian company Bemberg. As seen above, this yarn is not resorbable before oxidation. The fabric is thus knitted with this regenerated cellulose yarn, and is then submitted to a step of oxidation, in order to oxidize the cellulose and make this cellulose yarn resorbable. In the case of the fabric of FIGS. 2 to 4, the rib and the dense face will be in resorbable yarns after oxidation.

The needle bar of the knitting machine, corresponding to the yarns 4, is full-threaded. Such full threading allows better homogeneity and good density of the face. According to the weave described in FIG. 6, the yarns 4 are knitted according to the following scale: 1112/1110//.

In another embodiment of the invention, the dense face is knitted according to the weave shown in FIG. 7. In such a case, the needle bar of the knitting machine, corresponding to the yarns 4, is full-threaded and the yarns are knitted according to the following scale: 2223/1110//.

The three-dimensional fabric shown in FIG. 4 may thus be made by warp knitting of the four laps of yarns (1, 2, 3, 4) described above, according to the “cast-off stitch” technique on a Raschel knitting machine. Preferably, the various laps 1 to 4 are all knitted at the same time. Thus, the bonding yarns are distributed along the peripheral edges of the apertures of the porous face and extend substantially perpendicularly from this porous face to the dense face, making interstices for lateral interconnection with the other channels, and preventing bonding yarns from occupying an excessively large part of the volume of the transverse channels that are formed. The fabric may then be stabilized simply by baking at a temperature of between about 80° C. and 150° C. The thickness of the fabric obtained is from about 1.5 to 5 mm and has a weight of about 100 to 250 g/m².

In another embodiment of the invention, the three-dimensional fabric is made according to the “cast-on stitch” knitting technique.

The three-dimensional fabric according to the invention may also be made by weaving, according to the double-lap velour technique, as described, for example, in C. Villard's book “Manuel de théorie du tissage [Weaving theory manual]”, on page 229, Lyons, 1948.

The present invention will now be illustrated by the examples that follow.

### EXAMPLE 1

A knitted fabric containing three different types of yarn for the porous face, the rib and the dense face, respectively, is made on a Raschel knitting machine, according to the technique described in FIGS. 1 to 8 above.

The porous face is made of a non-resorbable polyethylene terephthalate multifilament yarn. The rib is made of polylactic acid (PLA) multifilament yarn. The dense face is made of regenerated cellulose multifilament yarn.

Once the knitted fabric has been made, it is subjected to a step of oxidation with NO₂.

This oxidation is performed by treating with NO₂ according to the teaching of patent U.S. Pat. No. 3,364,200. The NO₂ is dissolved in a non-aqueous solvent such as CO₂ or N₂ in gaseous, liquid or supercritical form, or in a liquid solvent, such as carbon tetrachloride or Freon 113, or perfluorinated substitutes thereof. The oxidation is followed by washing with the solvent, and then preferably by washing with isopropanol or acetone. The fabric is then dried under vacuum, and then cut into the form of reinforcing prostheses, which are packaged and sterilized with ethylene oxide.

According to this oxidation process, only the cellulose is oxidized. It becomes gradually water-soluble and resorbable after implanting the prosthesis into the patient's body.

A dipping of the prosthesis in water, just before implantation, may accelerate if necessary the conversion of the dense face into a continuous gel, the presence of this continuous hydrogel being desirable as soon as possible in order to avoid potential post surgical adhesions.

### EXAMPLE 2

A knitted three-dimensional fabric and knitted prostheses are made according to the method described in Example 1, the yarns used in Example 1 being replaced with the following yarns:
a monofilament polypropylene yarn for the porous face,
a regenerated cellulose yarn for the rib and the dense face.

The thus knitted fabric is then subjected to an oxidation step with NO₂. This oxidation is realised by reacting gaseous NO₂ in a concentration of 10 g/L, with a relationship of 1.3 gram of NO₂ per gram of cellulose. The reaction is continued for 4 hours. At the end of the reaction, a washing with an inert gas such as CO₂ or N₂ is done so as to remove the excess of NO₂. The fabric is then washed with a mixture isopropanol/water (1:1), and then with pure isopropanol. The fabric is then dried and cut under the form of reinforcement prosthesis which are then packaged and sterilised by gamma radiation.

### EXAMPLE 3

A knitted three-dimensional fabric and knitted prostheses are made according to the method described in Example 1, the yarns used in Example 1 being replaced with the following yarns:
a monofilament polypropylene yarn for the porous face,
a multifilament polyglycolic acid (PGA) yarn for the PGA rib.

The dense face is made of regenerated cellulose yarn as in Example 1.

The fabric thus knitted is then subjected to an oxidation as in Example 1.

### EXAMPLE 4

A knitted prosthesis is made on a Raschel knitting machine according to the knitting technique described in FIGS. 1 to 8 above, containing three different types of yarn for the porous face, the rib and the dense face, respectively.

The porous face is made of a non-resorbable polyethylene terephthalate multifilament yarn. The rib is made of polylactic acid (PLA) multifilament yarn. The dense face is made of PGA-oxidized cellulose as polyglucuronic acid composite yarn.

### EXAMPLE 5

A knitted prosthesis is made on a Raschel knitting machine according to the knitting technique described in FIGS. 1 to 8 above, containing three different types of yarn for the porous face, the rib and the dense face, respectively.

The porous face is made of a non-resorbable polypropylene multifilament and/or monofilament yarn. The rib is made of polylactic and glycolic acid (PLGA) multifilament yarn. The dense face is made of hyaluronate yarn.

### EXAMPLE 6

A knitted prosthesis is made on a Raschel knitting machine according to the knitting technique described in FIGS. 1 to 8 above, containing three different types of yarn for the porous face, the rib and the dense face, respectively.

The porous face is made of a non-resorbable polyethylene terephthalate multifilament yarn. The rib is made of polylactic acid (PLA) multifilament yarn. The dense face is made of polyglycolic acid multifilament yarn, impregnated with hyaluronate and/or with polyglucuronic acid.

### EXAMPLE 7

A fabric similar to that of Example 5 is made, in which the yarn of the dense face is impregnated with a mixture of hyaluronic acid and chitosan.

### EXAMPLE 8

A knitted prosthesis is made on a Raschel knitting machine according to the knitting technique described in FIGS. 1 to 8 above, containing three different types of yarn for the porous face, the rib and the dense face, respectively.

The porous face is made of a non-resorbable polypropylene monofilament yarn. The rib is made of polylactic acid (PLA) multifilament yarn. The dense face is made of polyglycolic acid (PGA) multifilament yarn, impregnated with hyaluronate according to the following method: the multifilament polyglycolic acid is impregnated with hyaluronate at pH 9, then crosslinked with heating with 1,4-butanediol diglycidyl ether, before knitting, at the time of drying with acetone of the PGA composite yarn.

In Examples 6, 7 and 8, the constituent impregnated yarns of the dense phase become highly hydrophilic at the surface and, for each of them, the chosen polysaccharide will be rapidly released or hydrated by a previous wetting and/or in the presence of moisture from the biological tissues, creating a viscous gel anchored in the stitches of the fabric according to the invention, by virtue of its viscosity and its crosslinking, if any. The spontaneous creation of this continuous gel gives the fabric according to the invention adhesion-preventing properties.

1. Three-dimensional prosthetic fabric comprising a first face and a second face, the said first face and the said second face being opposite each other and separated by the thickness of the said fabric, the said first face being porous, characterized in that the said second face is dense and is made of at least one first resorbable yarn.

2. Fabric according to claim 1, wherein the said first resorbable yarn consists of monofilaments and/or multifilaments of a biocompatible polymer material chosen from polyesters, polycaprolactones, polydioxanones, polyamides, polyethers and polysaccharides, and mixtures thereof.

3. Fabric according to claim 1, wherein the polyesters are chosen from polyhydroxy acids, preferably glycolic acid polymers, lactic acid polymers and hydroxybutyric acid polymers, and mixtures thereof.

4. Fabric according to claim 2, wherein the polysaccharides are chosen from hyaluronic acid, alginic acid, polyglucuronic acid, chitosan, starch and soluble cellulose derivatives, and mixtures thereof.

5. Fabric according to claim 4, wherein said polysaccharide is crosslinked.

6. Fabric according to claim 4, wherein the soluble cellulose derivatives are chosen from cellulose ethers, for instance carboxymethylcellulose, and oxidized celluloses, and mixtures thereof.

7. Fabric according to claim 6, wherein the oxidized celluloses are chosen from oxidized cellulose in which the C₆ primary alcohol is partially or totally oxidized to a carboxylic acid, for example, to give polyglucuronic acid, cellulose oxidized in the form of polyaldehydes with periodic acid, and cellulose of “viscose” type, manufactured from a solubilized and then regenerated and oxidized cellulose pulp, and mixtures thereof.

8. Fabric according to claim 7, wherein the said first resorbable yarn is a regenerated and oxidized cellulose multifilament yarn.

9. Fabric according to claim 3, wherein the said first resorbable yarn is a composite multifilament yarn of polyglycolic acid and of oxidized cellulose in polyglucuronic acid form.

10. Fabric according to claim 4, wherein the said first resorbable yarn is obtained by mixing a negatively charged polysaccharide, chosen from alginic acid, hyaluronic acid, polyglucuronic acid and mixtures thereof, and a positively charged polysaccharide, for instance chitosan.

11. Fabric according to preceding claim 1, wherein, since the said first resorbable yarn is a multifilament yarn composed of multiple filaments defining between themselves interstitial spaces, the said multifilament yarn is impregnated, in the said interstitial spaces, with a polysaccharide chosen from hyaluronic acid, alginic acid, polyglucuronic acid, chitosan, starch and soluble cellulose derivatives, and mixtures thereof.

12. Fabric according to claim 13, wherein the said first resorbable yarn is a multifilament yarn of polyglycolic acid impregnated with polyglucuronic acid.

13. Fabric according to claim 11, wherein the said first resorbable yarn is a multifilament yarn of polyglycolic acid impregnated with a mixture of polyglucuronic acid and chitosan.

14. Fabric according to claim 11, wherein the said first resorbable yarn is a multifilament yarn of polyglycolic acid impregnated with a mixture of hyaluronic acid and chitosan.

15. Fabric according to claim 11, wherein the said first resorbable yarn is a multifilament yarn of polyglycolic acid impregnated with hyaluronate crosslinked with 1,4-butanediol diglycidyl ether.

16. Fabric according to claim 11, wherein the said first resorbable yarn is a multifilament yarn of oxidized cellulose impregnated with chitosan.

17. Fabric according to claim 1, wherein the said first porous face is made of at least one second resorbable or non-resorbable yarn, consisting of monofilaments and/or multifilaments of a biocompatible polymer material.

18. Fabric according to claim 17, wherein the said second yarn is non-resorbable.

19. Fabric according to claim 18, wherein the said second yarn consists of monofilaments and/or multifilaments of a biocompatible polymer material chosen from polypropylene, polyethylene terephthalate, polytetrafluoroethylene, polyamide, polyvinyl-difluorene, and mixtures thereof.

20. Fabric according to claim 1, wherein the said first face and the said second face are linked together via a rib made of at least one third resorbable or non-resorbable yarn, consisting of monofilaments and/or multifilaments of a biocompatible polymer material.

21. Fabric according to the claim 20, wherein the said third yarn is resorbable.

22. Fabric according to claim 21, wherein the said third yarn consists of monofilaments and/or multifilaments of a biocompatible polymer material chosen from polyesters, polycaprolactones, polydioxanones, polyalkanoates, polyamides, polyphosphazenes, polyacetals, polyurethanes, polyorthoesters, polycarbonates and polyanhydrides, and mixtures thereof.

23. Fabric according to claim 22, wherein the polyesters are chosen from polyhydroxy acids, preferably glycolic acid polymers, lactic acid polymers and hydroxybutyric acid polymers, and mixtures thereof.

24. Fabric according to claim 23, wherein the said third yarn is a polylactic acid multifilament yarn.

25. Fabric according to claim 23, wherein the said third yarn is a polyglycolic acid multifilament yarn.

26. Fabric according to claim 23, wherein the said third yarn is a polylactic and glycolic acid multifilament yarn.

27. Fabric according to claims 1, wherein the said dense face comprises at least one lap of first close-knitted yarns which determines a unified but permeable face.

28. Fabric according to claim 17, wherein the said porous face comprises at least two laps of knitted second yarns, determining hexagonal-shaped apertures.

29. Fabric according to claim 20, wherein the said rib comprises at least one lap of third yarns, extending substantially perpendicularly from the porous face to the dense face, the said third yarns being distributed, on the said porous face, along the peripheral edges of the said hexagonal apertures.

30. Fabric according to claim 29, wherein the said third yarns form substantially parallel transverse channels, the internal cross section of which is free of yarns, the said channels emerging on either side of the fabric, on the porous face according to the said hexagonal apertures, and on the dense face, respectively.

31. Fabric according to claim 30, characterized in that the mean diameter of the transverse channels is greater than or equal to 0.3 mm, preferably ranging from 0.7 to 3 mm and more preferably ranging from 1.3 to 1.7 mm.

32. Fabric according to claim 31, wherein the transverse channels have a length, corresponding to the thickness of the said fabric, ranging from 0.5 to 5 mm and preferably ranging from 1.5 to 3 mm.

33. Fabric according to claim 30, wherein the rib determines for each channel a porous inner wall interconnecting with the neighbouring channels, the said porous inner wall defining interstices for passing between channels.

34. Fabric according to claim 33, wherein the said interstices for passing between channels have a width ranging from 100 to 300 microns.

35. Prosthesis for reinforcing, protecting or supporting a tissue wall, wherein it is obtained by cutting out from a prosthetic fabric according to claim 1.

36. Process for preparing a three-dimensional prosthetic fabric comprising a first face and a second face, the said first face and the said second face being opposite each other and separated by the thickness of the said fabric, the said first face being porous, the said second face being dense and resorbable, comprising a step of manufacturing a three-dimensional knitted fabric on a warp knitting machine or a Raschel knitting machine in at least one lap of yarns defining the porous face, at least one lap of yarns defining the thickness of the said fabric, and at least one lap of yarn defining the said dense face, characterized in that the said lap defining the said dense face is obtained using a full-threaded guide bar with at least one first resorbable yarn.

37. Process for preparing a three-dimensional prosthetic fabric comprising a first face and a second face, the said first face and the said second face being opposite each other and separated by the thickness of the said fabric, the said first face being porous, the said second face being dense and resorbable, comprising a step of manufacturing a three-dimensional knitted fabric on a warp knitting machine or Raschel knitting machine in at least one lap of yarns defining the porous face, at least one lap of yarns defining the thickness of the said fabric, and at least one lap of yarn defining the said dense face, wherein:
in a first step, the said lap defining the said dense face is obtained using a full-threaded guide bar with at least one first regenerated cellulose yarn, and
in a second step, the said fabric is subjected to an oxidation step.

38. Process according to claim 39, wherein the said fabric is subjected to oxidation with sodium periodate or with periodic acid and is then rinsed in an aqueous acetone or alcohol solution, and washed with pure acetone or alcohol before drying.

39. Process according to claim 37, wherein the said fabric is subjected to oxidation with gaseous nitrogen dioxide at a temperature ranging from 20 to 50° C., in particular from 25 to 40° C.

40. Process according to claim 37, wherein the said fabric is subjected to oxidation with gaseous nitrogen dioxide for a time ranging from 2 to 48 hours, in particular from 3 to 8 hours.

41. Process according to claim 39, wherein the nitrogen dioxide concentration is less than 14 g/L, in particular ranges from 6 to 12 g/L.

42. Process according to claim 39, wherein the weight ratio NO₂/cellulose is greater than 0.9.

43. Process according to claim 37, wherein the said fabric is subjected to oxidation with gaseous nitrogen dioxide in the presence of air or oxygen.

44. Process according to claim 37, wherein after oxidation with nitrogen dioxide, said fabric is subjected to a washing step with an inert gas like CO₂ or N₂ in order to remove the excess of NO₂, followed by a washing with a volatile alcohol.

45. Process according to claim 37, wherein the said fabric is subjected to oxidation with nitrogen dioxide in a suitable non-aqueous solvent and is then washed with an aqueous isopropanol solution.

46. Process according to claim 44, wherein a plasticizer, such as glycerol or polyethylene glycol, is added to the washing step.

47. Process according to claim 36, wherein the said first yarns of the lap defining the dense face are knitted according to the scale 1112/1110//.

48. Process according to claim 36, wherein the said first yarns of the lap defining the dense face are knitted according to the scale 2223/1110//.

49. Process according to claim 37, wherein, before oxidation, the fabric is stabilized by baking at a temperature ranging from 80 to 150° C.
